(19) United States
(12) Patent Application Publication
(10) Pub. No.: US 2016/0166192 A1
(43) Pub. Date: Jun. 16, 2016

(54) MAGNETIC RESONANCE IMAGING TOOL TO DETECT CLINICAL DIFFERENCE IN BRAIN ANATOMY

(71) Applicant: CHILDREN'S HOSTPITAL LOS ANGELES, Los Angeles, CA (US)

(72) Inventors: Natasha Lepore, Los Angeles, CA (US); Fernando Yepes-Calderon, Los Angeles, CA (US); Yalin Wang, Tempe, AZ (US); Paul Thompson, Redondo Beach, CA (US); Xavier Pennec, Antibes (FR); Marvin Nelson, La Cañada Flintridge, CA (US); Caroline Brun, New York, NY (US)

(73) Assignee: Children's Hospital Los Angeles, Los Angeles, CA (US)

(21) Appl. No.: 14/908,499

(22) PCT Filed: Jul. 30, 2014
(86) PCT No.: PCT/US14/48973

§ 371 Date: Jan. 28, 2016

Related U.S. Application Data
(60) Provisional application No. 61/859,807, filed on Jul. 30, 2013.

(51) Int. Cl. A61B 5/00; A61B 5/055

(52) U.S. Cl. CPC A61B 5/4064; A61B 5/0042; A61B 5/055; A61B 5/7246; A61B 5/4836

(57) ABSTRACT

The present invention provides a system providing a suite of functions for automatically analyzing MRI images of brains to quantitatively assess brain development in children and adolescents. The system compares MRI images of a subject's brain to a database of normal brains, identifies regions showing abnormal growth, structure, and/or morphology, and notifies the health care provider of such abnormalities. The health care provider is notified by way of a graphical image depicted on a display highlighting the abnormal area(s) of the brain.

### PRIORITY

The present application claims priority to U.S. Provisional Application 61/859,807, filed on Jul. 30, 2013, which is hereby incorporated by reference in its entirety.

### COPYRIGHT

A portion of the disclosure of this patent document contains material that is subject to copyright protection. The copyright owner has no objection to the facsimile reproduction by anyone of the patent disclosure, as it appears in the Patent and Trademark Office patent files or records, but otherwise reserves all copyright rights whatsoever.

### TECHNICAL FIELD

The present invention relates to the field of medical and clinical tools for detection of morphology of the brain. More specifically, the invention relates to a Magnetic Resonance Imaging (MRI) analysis tool, and a method of using that analysis tool to detect clinically relevant anomalies in brain anatomy and brain development of subjects. The invention has particular utility in detecting differences in brain anatomy of the developing brains of children.

### BACKGROUND

Magnetic Resonance Imaging (MRI) is a safe and non-invasive tool to visualize organs in vivo. In particular, in fetuses, neonates, and children, MRI plays a key role both for clinical diagnostics and for providing insights about development in health and disease through medical research. Diagnosing brain abnormalities early in development is of extreme importance to a child's health and to society in general. Apart from the obvious emotional toll that brain disorders inflict on patients and families, anomalies in development may result in long-term impairment, including for example, cognitive or motor delays. Therefore, their cost to society needs to be added over a lifetime. The World Health Organization has recognized this importance and named early child development one of the top priority health targets of the century.

Magnetic Resonance Imaging has become a common, though specialized, diagnosis tool. For example, Children's Hospital Los Angeles performed 6609 MRI acquisitions in children for the period of July 2012 to January 2013, compared with 5822 acquisitions done in the same period of the previous year (July 2011-January 2012), an increase of 13.6%. Among the 6609 acquisitions, 1528 were performed on hospitalized patients and 5081 on out-clinic patients. It is clear to practitioners in the art that this upward trend will continue to increase in the coming years as MRI costs decrease and analyses and scanning protocols improve.

Despite the widespread use of clinical MRI, there is a shortage of quantitative analytical tools in the clinic, and this is particularly true of tools for pediatric scans. The vast majority of diagnoses are done using visual inspection of images by neuroradiologists who have differing levels of experience and expertise. Because the diagnoses rely on radiologists' interpretations of MRI images, these diagnoses depend substantially on the radiologist's expertise and some of them may miss subtle brain anomalies. This is particularly worrisome in hospitals that do not explicitly specialize in children care, and thus might have widely different levels of expertise among their radiologists.

Certain software is available for MRI image analysis. One of the leading publicly available software packages for brain MRI analysis is the FreeSurfer brain imaging software originally developed at the Massachusetts General Hospital. In children, the FreeSurfer software predicts age in children by using an analysis algorithm to combine a set of 231 biomarkers and output an age prediction. However, that software is geared toward researchers rather than clinicians and the software has been limited to children above three years old. Furthermore, while the software has many uses, the only output of this particular analysis is the predicted age of the child based on the biomarkers. One clinical derivative of Freesurfer is NeuroQuant®, which is one of few commercial tools capable of mapping brain changes in adults for Alzheimer's disease and dementias. However, NeuroQuant® calculates whole volumes of structures instead of fine-grained 3D maps and is not applicable to young children. Another available software package is MIM neuro, which generates comparisons to normal in PET and SPECT brain images.

Other publicly available software for brain researchers includes the MRVision visualization software for MRI analyses with simple image processing tools. It can perform basic computations on images but cannot, for example, provide comparison to a statistical distribution of normal subjects, which is essential to diagnose deviations from normal. The MIPAV software tool is another sophisticated image analysis tool for medical imaging data. It has a set of general image processing options, particularly for region of interest (ROI) analyses, and some more specialized ones for particular applications, none of which focus on children. Yet another tool is the Analyze software from the Mayo Clinic. It can be used to perform image segmentation, image fusion, visualization, and ROI analyses of multi-modality images. However, this software is not geared toward automatic processing of child development and lacks both the comparison to normal tool of the present invention and automated quantitative comparisons of ROIs to normal children.

Numerous general MRI processing tools created for researchers also exist. Examples of such packages include: FSL, SPM, MedInria, Camino, Trackvis, Maracas, BBTK, Brainsuite, ImageJ, and ITK-SNAP. These packages implement cutting edge algorithms and are typically offered free of charge. SliceOMatic is a commercial research tool that performs MRI image segmentations and measures volumes of structures, but does not compare them to normal subjects. Analyses are done by comparing scalar volumes, rather than using the multivariate complete shape information (directional volume changes). However, the end-user must have a high level of technical skill in order to use such tools effectively. Furthermore, these platforms do not target specific clinical necessities, as they are created to assist researchers for image preprocessing, statistical analysis, and visualization of results.

Commercial software is available for image processing. For example, Santesoft has established its niche in the DICOM format. Services include data compression, anonymization, and visualization as well as functionalities to create videos and graphical information in portable formats. As another example, Syntermed is a company that develops medical software to meet physician necessities. Their primary niche is in cardiac applications, but recently, this company launched the product NeuroQ, software that provides a basic functionality for visualization and reconstruction of positron emission tomography (PET) images.

However, currently there is no FDA approved software to quantitatively evaluate whether different regions of a child's brain are developing normally. When abnormalities are suspected, they are currently evaluated through visual inspection of MRI images. However, as an abundance of studies have shown, many anatomical anomalies may only be assessed through numerical comparisons with a healthy subject population. Thus, new clinical software is urgently needed to address the needs of children. There is a need for MRI image analysis tools to assist physicians in the study, diagnosis, and prognosis of the developing brain via quantitative analysis. There is also a need for a system to visually display anomalies between an MRI brain image and a normal brain.

### SUMMARY

According to one example, a method for analyzing a brain of a subject is disclosed. MRI image data of the brain or portion of the brain of the subject is acquired. One or more morphological characteristics of one or more structures of the brain are determined. The one or more morphological characteristics are compared to the same one or more morphological characteristics of the structure of a normal brain. A quantitative value of the difference between the determined one or more morphological characteristics and the same one or more morphological characteristics of the normal brain is calculated. The quantitative value is output in a format for a health care provider.

Another example is a system for analyzing a brain of a subject. The system includes a storage device including MRI image data of the brain or portion of the brain of the subject. A database including data from a normal brain is included. A controller is coupled to the storage device and the database. The controller determines one or more morphological characteristics of one or more structures of the brain. The controller compares the one or more morphological characteristics to the same one or more morphological characteristics of the structure of a normal brain from the data from the normal brain. The controller calculates a quantitative value of the difference between the determined one or more morphological characteristics and the same one or more morphological characteristics of the normal brain.

Another example is a method of treating a subject having a developmental disorder of the brain. MRI image data of the brain or portion of the brain of the subject is acquired. One or more morphological characteristics of one or more structures of the brain is determined. The one or more morphological characteristics is compared to one or more of the same morphological characteristic of the structure of a normal brain. A quantitative value of the difference between the determined one or more morphological characteristics and the one or more same morphological characteristics of the normal brain is calculated. Based on the quantitative value, it is determined that the subject's brain is undergoing, or has undergone, a developmental disorder. The patient is treated to reduce or eliminate the clinical effects of the disorder.

Another example is a non-transitory, machine readable medium having stored thereon instructions for analyzing a brain of a subject, the stored instructions comprising machine executable code, which when executed by at least one machine processor, causes the machine to acquire MRI image data of the brain or portion of the brain of the subject. The instructions cause the machine to determine one or more morphological characteristics of one or more structures of the brain. The instructions cause the machine to compare the one or more morphological characteristics to the same one or more morphological characteristics of the structure of a normal brain. The instructions cause the machine to calculate a quantitative value of the difference between the determined one or more morphological characteristics and the one or more morphological characteristics of the normal brain. The instructions cause the machine to output the quantitative value in a format for a health care provider.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1 is a block diagram of an example MRI image data analysis system;

FIG. 2 is a screen image of a user interface of the data analysis system in FIG. 1;

FIG. 3A is a screen image of a three-dimensional image of the whole brain from the system in FIG. 1;

FIG. 3B is a screen image of a comparison of a conventional two-dimensional image with a three-dimensional image of a corpus callosum area from the system in FIG. 1;

FIG. 3C is a screen image of another three-dimensional image of sub-cortical brain structures for diagnosis from the system in FIG. 1;

FIG. 3D is a screen image of a plots of data for diagnosis of hydrocephalus from the system in FIG. 1;

FIG. 4 is a block diagram of a computational device which may be incorporated in the system in FIG. 1; and

FIG. 5 is a flow diagram of the process followed by the system in FIG. 1 to analyze MRI image data.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

The present invention addresses the need in the art for MRI image analysis tools to assist physicians in the study, diagnosis, and prognosis of the developing brain by providing such tools. The invention provides an important new tool to physicians and neuro-radiologists in particular, and to hospitals and clinics in general, and particularly those that cater to children.

In a first general aspect of the invention, software for analyzing MRI image data is provided in conjunction with an analysis system. The software of the present invention provides computer-implemented methods that provide some or all of the following features: 1) analysis of differences in brain morphology between a subject and a group of subjects taken from brain MRI image data; 2) determination of shape differences throughout the brain volume and in particular regions or structures such as on the surface of the cortex or on the surface of subcortical structures; 3) generation of basic values, such as head and brain circumferences, whole brain volume, and white and grey matter volumes; 4) a comparison framework based on a dynamically-created “gold” standard in which the gold standard is created by age and metrics grouping, allowing inferences about the state of development of the brain; 5) visualization of brain MRI images with highlighted abnormal regions, as compared to healthy subjects of the same age or same anatomical metrics; and 6) production of a quantitative report of detected anomalies Importantly, the software of the invention may be used to analyze MRI image data for brains from infants to 18 year old patients or older.

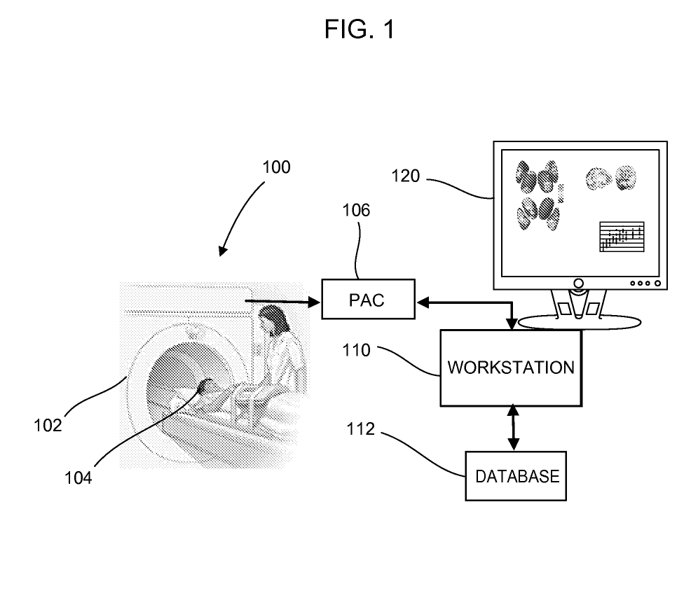

FIG. 1 shows an example of an MRI image data analysis system 100. The system 100 includes an MRI device 102, which takes MRI image data of the brain of a subject 104. In this example, the subject 104 is a child, but other subjects of any age may be treated with the assistance of the system 100. In this example, the MRI device 102 is an MRI scanner. The output image data of the MRI device 102 is stored in a memory device either locally or remotely such as in a hospital picture archiving and communication (PAC) system 106. The PAC system 106 is accessible via a network to a workstation 110. Alternatively, the output image data and other subject data may be stored in a memory directly accessible by the workstation 110. The workstation 110 is capable of operating the MRI analysis software for analyzing MRI image data of the subject 104 from the PAC system 106. The workstation 110 is also coupled to a database 112, which includes image data and quantitative data of the brains of healthy children previously imaged and obtained. The data may be organized in groups of children by age or other criteria such as gender and ethnicity. In this example, the database 112 may be stored on the workstation 110 or remotely such as in the PAC system 106 or in another network accessible node. The analysis software produces visual outputs reflecting the quantitative value of the difference between at least one morphological characteristic of the brain to the mean of the same at least one morphological characteristic of a normal brain or a distribution of normal brains and the standard deviation from the mean in a format for a health care provider through a display 120.

The MRI analysis software operated by the workstation 110 includes a set of algorithms to analyze differences in brain morphology between groups of subjects from brain MRI images, including children 0-18 years old. The algorithms are herein combined to enable comparisons to a large data set of healthy children, to determine how targeted brain structures develop in a normal population. For example, an initial data set may be obtained from Dr. Sean Deoni's database at Brown University of children aged 0-5 years old. Another initial data set is the NIH Pediatric Development database, an MRI study of about 500 children aged 0-18 years old. This data serves to build a distribution on normal variation in brain anatomy per gender and age group to which individual patients can be compared. The MRI analysis software allows for updates such that, as new data are obtained from new healthy patients, the distribution on normal may be refined with the new data. The database 112 is therefore updated if the data from the subject such as the subject 104 is within an acceptable range of the previous normal. For example, data from the subject may be added to update the database 112 if there are no abnormally developing regions (outside of one standard deviation from normal for example), and if subsequent medical visits confirm this diagnosis.

This aspect of the invention provides software for a clinic to detect anomalies in the brain morphology of children. Conveniently, this aspect of the invention, and the methods of diagnosing and treating discussed below, can be practiced on patients who are already getting an MRI prescribed by their physicians as part of their medical care. In essence, the MRI analysis software of the invention takes the input of the brain MRI of a patient such as the subject 104 in FIG. 1, and compares it to that of a normal population of the same age/gender, in order to pinpoint regions of the patient's brain that are not following a standard developmental trajectory. Doctors need to make comparisons between the brains of their patients and that of healthy subjects daily, and the present invention provides a basic reference tool to make such comparisons quantitatively, rather than qualitatively, as is the current practice. These software tools of the MRI analysis software greatly impact clinical practice in several ways, including shortening diagnosis time, improving detection and quantification, providing a comparison to a healthy population of the same age/gender, and the ability to better assess developmental changes over time. Additionally, clinicians and scientists can benefit from past experiences through the capability of retrospective querying software results, and correlation with diagnosis and prognosis.

In an exemplary embodiment of the invention, the MRI analysis software running on the workstation 110 includes computer implemented code for: a) acquiring MRI image data relating to the brain of a subject; b) determining one or more morphological characteristics of one or more structures of the brain; c) comparing the one or more morphological characteristics to at least one morphological characteristic of the structure of a normal brain; d) calculating a quantitative value of the difference between the determined one or more morphological characteristics and the at least one morphological characteristic of the normal brain; and e) outputting the quantitative value in a format for a health care provider. In this example, the subject is a human, such as a child of age 1 day to 18 years (or older, such as up to 21 or 22 years).

The software may acquire MRI image data relating to the brain of a specific subject such as the subject 104 from a memory device such as the hospital picture archiving and communication (PAC) system 106 of the brain or portion of the brain of a subject such as the subject 104 in FIG. 1. In this example, this is accomplished by using a non-conventional port of the workstation 110 such as a TCP connection where the data only flows after a rigorous authentication. Authentication may be achieved via encryption and tokenized security or other methods.

The MRI analysis software may determine morphological characteristics of selected structures of the brain. Whole brain analysis is done automatically, and if after examination, the user wants more information on a particular structure, this structure is analyzed separately in more detail. Morphological characteristics include volume differences and their direction in the whole brain and, when zooming in on structures, surface features such as area differences and their direction, as well as thickness at each point of the structure. Other characteristics include the head circumference, grey matter volume, white matter volume, and ventricular volume. The structures may be automatically selected by the MRI analysis software and presented for individual presentation to the user via a menu or other selection devices. Alternatively, the user may select different brain structures for more detailed images from a general brain image.

The MRI analysis software may compare the selected structures of the imaged brain with those of a normal brain. Determining morphological characteristics and comparing the selected structures are accomplished using routines developed to look at shape differences throughout the brain volume, on the surface of the cortex, and on the surface of subcortical structures. Given T1-weighted three-dimensional magnetic resonance images, which can be acquired rapidly on any commercial 1.5T or 3T MRI scanner the MRI analysis software determines deviation from age- and sex-matched healthy normal standards in brain structure. Measurements are generated both as numerical values and as a color map indicating differences from normal, as described in the table below.

| METRIC | QUANTITATIVE MEASUREMENTS |
|---|---|
| Head circumference | Value + deviation from normal for patient's |
| | age/gender |
| Total white matter volume | Value + deviation from normal for patient's |
| | age/gender |
| Total gray matter volume | Value + deviation from normal for patient's |
| | age/gender |
| Ventricular CSF volume | Value + deviation from normal for patient's |
| | age/gender |
| Whole brain morphometry | 3D maps of deviations from normal |
| Subcortical morphometry | 3D maps of subcortical structures + |
| | deviation from normal for patient's |
| | age/gender |

The MRI analysis software may calculate a quantitative value of difference and number of standard deviations from normal between the morphological characteristic(s) using the same routines. The quantitative values may be expressed numerically or graphically as will be explained below. The quantitative difference values are stored along with patient data in a memory system such as the PAC 106 in FIG. 1.

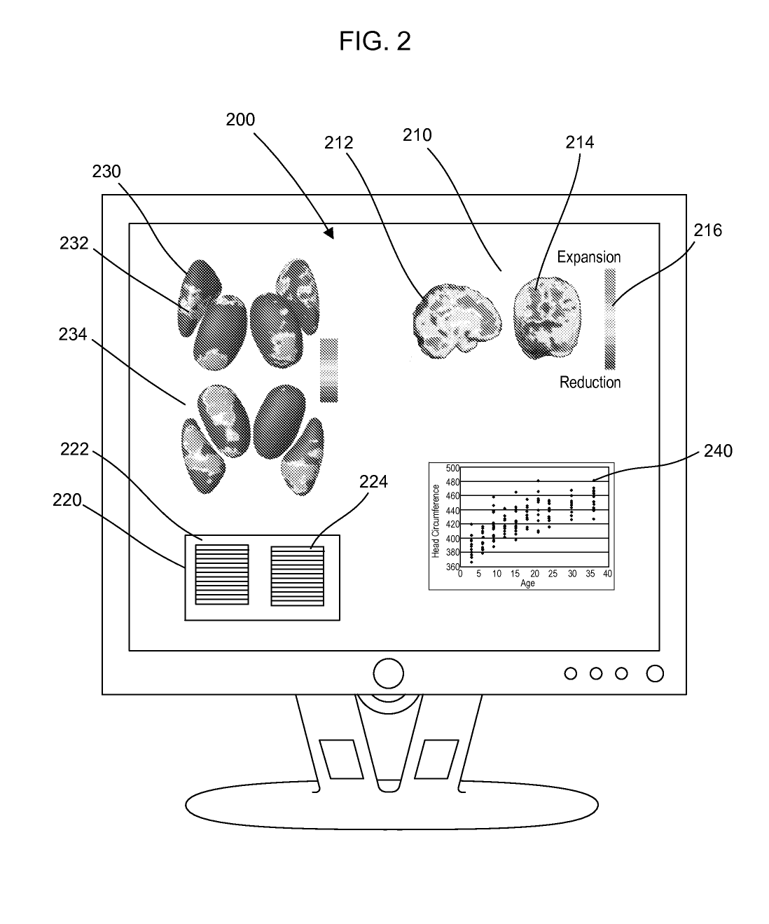

The MRI analysis software may output the quantitative value(s) in a format that is useable for a human health care provider. An example of the output is accomplished through a graphical user interface 200 specifically geared toward clinicians as shown in FIG. 2.

FIG. 2 is an example of a graphical user interface 200 that may be output on the display 120 in FIG. 1. Of course, the interface 200 may be displayed on other display devices and made available to users. In preferred embodiments, outputting the quantitative value(s) includes providing the graphical user interface (GUI) 200 that displays areas of the patient's brain that are developing abnormally to a user. For example, the output may be a two-dimensional or three-dimensional image of the patient's whole brain showing in highlighted form one or more areas of the brain that are abnormal in size and/or shape. This output allows the physician to quickly focus on areas of interest without the need for the physician to review in detail the entire MRI image, as is currently practiced. In this example, the interface 200 includes a three-dimensional whole brain display area 210 that includes a side view 212 and a top view 214 of a patient's whole brain. The brain images in the side view 212 and the top view 214 includes highlighted areas which indicate abnormal structures such as for example the putamen, thalamus, hippocampus, ventricles, caudate or corpus callosum. The highlighted areas may be color coded according to a color scale 216 that indicate the value of deviation from a normal brain or distribution of normal brains. The interface 200 also includes an area 230 for display of specific structures of the brain, a table 220 for output display of numerical values obtained from the MRI image, and a plot 240 for charting the values in comparison to a normal brain. Of course, other means for outputting data known in the art are encompassed and envisioned by this disclosure.

In non-limiting exemplary embodiments, the MRI analysis software outputs basic values from a brain image, such as head and brain circumferences, whole brain volume, grey matter and white matter volumes and ventricular volumes, and by what amount these values differ from those of other children of the same age. Such values may be displayed in the table 220 which is part of the interface 200. The table 220 includes a first column 222, which includes values determined from the MRI image of the subject 104 in FIG. 1 and a second column 224, which includes the amount that such values differ from those of children of the same age.

These measurement values are used daily in the clinic, and the ordinary artisan will immediately recognize that being able to automate and standardize the process of outputting these values to the physician is of great help in achieving a rapid, accurate evaluation of the state of development of the subject's brain. In non-limiting exemplary embodiments, the software outputs an indication of regions of the subject's brain where the volume of brain regions and the ventricles are altered compared to healthy subjects of the same age. This may be shown graphically in the area 230 for specific structure of the brain. The statistical analysis for this process consists of: 1) registering the subject's data to an average brain; 2) computing statistics on the Jacobian of the deformation at each voxel from the registration; and 3) comparing the subject's results to a distribution of data in healthy children of the same age and gender, to see whether the subject's data falls within the normal range of the distribution.

In non-limiting exemplary embodiments, the MRI analysis software of the invention is capable of determining regional differences in surface area and thickness of subcortical structures and outputting those differences. The area 230 in FIG. 2 shows an example close-up isolation of selected brain structures. In this example, the area 230 shows some subcortical structures from a top view 232 and a bottom view 234 and are maps showing abnormal regions in red, based on measurements of directional surface area and thickness. Of course other views may be displayed, and other specific brain structures may be shown in the area 230 and other colors may indicate abnormal regions. Similar to the images shown in the whole brain display area 210, the structures displayed in the area 230 may be color coded to indicate the number of deviations from the normal.

In these embodiments, regions throughout the brain volume whose morphology deviates from that of the healthy group are identified using a powerful statistical method performed by the MRI analysis software described above. Such an output may be plotted on a graph for visual display such as the graph 240 in FIG. 2.

In highly preferred embodiments, the MRI analysis software of the invention provides the practitioner with a detailed, graphical image of a subject's brain, or portion thereof, that highlights brain regions or structures that are developing abnormally. The software of the invention thus can help in differentiating normally and abnormally developing children's brains.

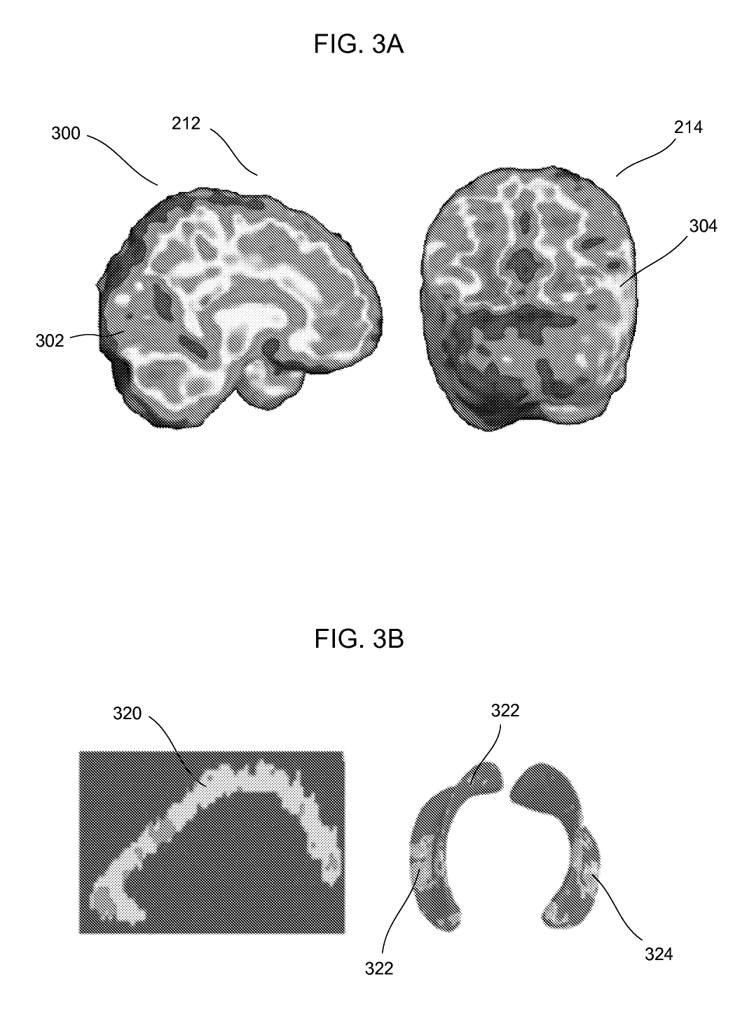

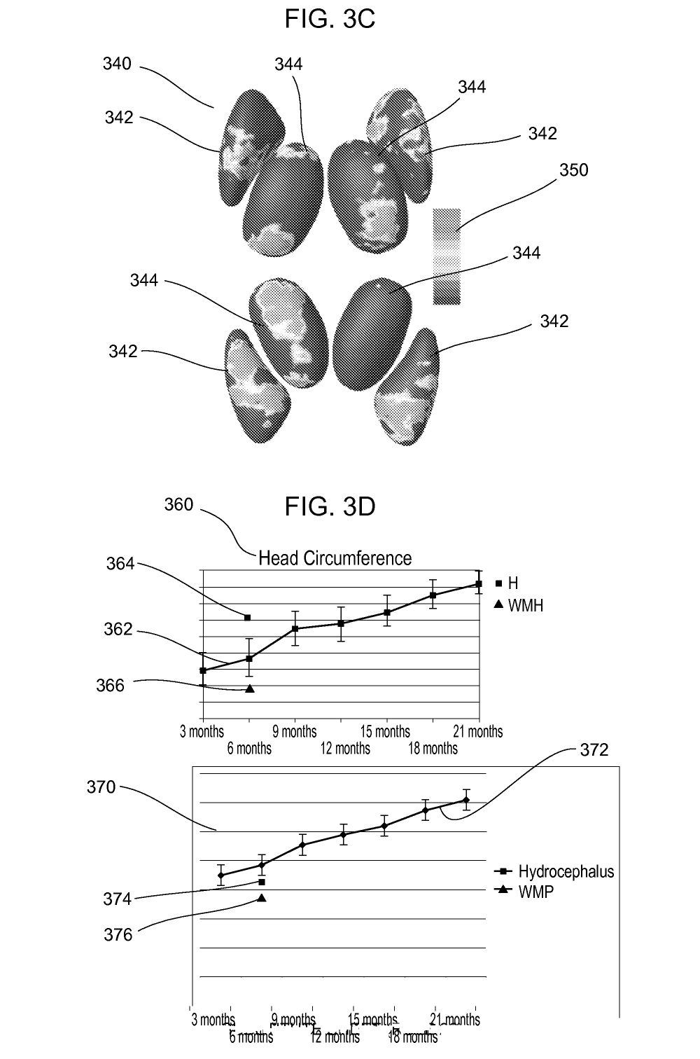

FIG. 3A is a close up image 300 of the whole brain display area 210 in FIG. 2 including a side view 212 and a top view 212. The close up image 300 shows the distribution of volume differences across the brain compared to a normal brain or distribution of normal means. In this example, a three dimensional section 302 of a side view of the while brain and a three dimensional section 304 of a top view of the brain is shown with colors indicating regions of increases and decreases compared to normal.

FIG. 3B is a comparison between an image 310 of a brain structure from current MRI images to an image of the same brain structure 320 using the present software. The brain structure image 320 may be displayed in the area 230 in FIG. 2. Compared to standard research-based analyses of brain structure, which compute changes in brain anatomy using only part of the shape difference information as in the image 310, the software captures full three-dimensional regional differences in shape as shown in the three-dimensional image 320. In this example, the image 310 is a standard 2D analysis slice of the corpus callosum area of the brain. In contrast, the MRI analysis software displays a full three dimensional image 320 of the corpus callosum area. In this example, different colors may represent comparisons to the normal. Of course other data such as the differences to the mean of the normal distribution or the standard deviation. The three-dimensional image 320 includes regional differences in shape represented by areas 322 and 324 to a normal brain.

FIG. 3C is a close up image 340 of the display area 230 in FIG. 2. The image 340 is a subcortical structure from the whole brain image in the whole brain area 210 in FIG. 2. In this example, the image 340 includes the top view 232 and the bottom view 234 of the subcortical structure. In this example, the subcortical structure shows the right and left putamen 342 and the right and left thalamus 344. The images of the putamen 342 and the thalamus 344 are color coded. In this example, a color scale 350 represents statistical deviations from normal. Of course other types of scales may be represented. These subcortical structures are implicated in many pediatric brain diseases such as ADHD. Determining regions of abnormal growth help clinicians generate an initial diagnostic and design targeted therapies, while follow-up examinations using the present MRI analysis software will assist the physician in determining whether an implemented treatment regimen is working.

FIG. 3D shows a plot 360 of brain circumference and a plot 370 of white matter volume which may be displayed in the plot area 240 in FIG. 2. The plot 360 includes a line 362 which shows the normal brain circumference. A point 364 shows the circumference of a subject with hydrocephalous while a second point 366 shows the circumference of a subject with white matter hypoplasia. Similarly, the plot 370 includes a line 372 which shows the normal white matter volume level. A point 374 shows the volume of a subject with hydrocephalous while a second point 376 shows the volume of a subject with white matter hypoplasia. Both points show that white matter volume is below the normal for both conditions.

Within the context of the present invention, a physician might need for example to determine whether a newborn has hydrocephaly or white matter hypoplasia, which requires comparing ventricular and white matter thicknesses. For example, it is difficult to assess the overall size of the brain when the display is always enlarged. When the brain is microcephalic, the ventricles will be enlarged due to abnormally developing white matter, but when enlarged to fill the frame of the viewing program, may be confused with hydrocephalus. Likewise, when the lateral ventricles are enlarged, for example due to diseases that affect the surrounding tissues, the CSF volume goes up, but it is difficult to assess by visual qualitative inspection alone whether grey and white matter volumes have increased and how tissues change after some form of treatment has been done. Using known approaches, these determinations are done visually from an MRI image and are prone to significant errors that can generate erroneous diagnoses, and hence affect follow up treatment. Treatment for hydrocephaly, for example, requires the insertion of a shunt to remove excess cerebrospinal fluid (CSF), while in white matter hypoplasia, the white matter is not developing normally, but CSF is produced at an appropriate rate. More accurate comparisons through the MRI analysis software producing the plot 360 showing a larger circumference in point 364 indicating hydrocephalous to assist in correct diagnosis of ventricular and white matter thicknesses deviating from normal levels thereby preventing unnecessary invasive treatment to reduce CSF levels. In contrast, subjects with white matter hypoplasia have a smaller than normal circumference as shown by the point 366 and smaller white matter volume as shown by the point 376.

In an exemplary embodiment of the invention, data is gathered from the CHLA database on children with hydrocephaly and white matter hypoplasia and stored in the database 112. Both groups of children get regular MRI scans (every few months) as well as follow-up care. Data labels are removed prior to analysis of the scans, and the software is used to distinguish between the two disorders at an earlier age than visual inspection by CHLA radiologists, who are highly skilled, being dedicated to treatment of children.

Furthermore, as an abundance of studies have shown, many morphological anomalies may only be assessed through numerical comparisons with a normal population. The present invention thus is of significant importance to radiology departments that evaluate child brain MRIs, both in general hospitals and in those that cater primarily to children. While the invention serves all radiologists, it is particularly useful to physicians who see fewer children to help compensate for reduced experience with this population. The present MRI analysis software is an assisting tool. However, the concepts described above may be incorporated into a stand-alone diagnostic and prognostic tool or may be used as a first-level diagnostic and/or prognostic tool, serving as a starting point for radiologists and doctors to evaluate potential developmental disorders of the brain.

Among other things, the present invention provides the following advantages, desired in the art for years, over previous technologies:

Quantification of developmental abnormalities in the brain;

Visualization of indexes and measurements in previous subjects with similar symptoms;

Visualization of indexes and measurements in previous subjects with similar diagnoses;

The ability to determine whether a subject is achieving developmental milestones even in the absence of known pathologies;

The creation of a population of normal to which individual children's images can be compared;

Computer assisted reporting;

Visualization tool for clearer evaluation;

Data standardization for retrospective querying;

Capability of morphometric analysis of brain structure using the complete shape information;

Case study sharing;

Capacity to integrate customized pipelines of analysis and image treatment;

Evolutionary comparison via regular scanning during treatment, so patients are not only profiled by their actual state but also by the way they reach it; and

Capacity to predict quantitative and qualitative outcomes based in previous experiences.

The software of the invention are an integrated suite or package of brain MRI analysis algorithms that provides a tool for the assessment of brain development in children, comparison to a normal population, and visualization of areas of potential concern. The MRI analysis software can be used in all patients who have already been prescribed an MRI by their physician thereby producing MRI image data, or can be used specifically for the purposed disclosed herein. Physicians routinely prescribe obtaining MRI images as a safe imaging procedure in children with suspected brain injury or abnormality. Good visualization of brain structure is often essential in diagnosing brain disease, but other imaging methods for structural imaging, such as CT, involve ionizing radiation which is particularly harmful to children. MRI is routinely prescribed, for example, in patients with congenital heart disorders, seizures, Fragile X syndrome, prematurity, hydrocephaly, cancer, unspecified neurodevelopmental delays, and other diseases and disorders. In MRI post-processing research, subject inter-variability is typically controlled through building a distribution of normal—using brains scans of healthy subjects—to which the patient data is compared. In other words, the collected data serves to build a distribution on normal variation in brain anatomy per gender and age group.

It will be understood by the skilled artisan that MRI post-processing analyses in children poses many challenges. The brain exhibits different image contrasts at different stages of development, and size and shape of structures vary rapidly. Additionally, motion artifacts can be quite severe in non-sedated children who are too young to stay still when asked, requiring the use of shorter scanning sequences that might affect image quality. Finally, data on healthy children is scarce, as parents are often reluctant to subject their healthy child to an MRI. As a result, the majority of MRI research has targeted older children and adults. Recently, the advent of new scanning protocols and more sophisticated post-processing tools have allowed researchers to study younger populations. However, much remains to be done on the post-processing side, and few image analysis tools have been built with an eye to translation to the clinic. The present invention recognizes the deficiencies in the art and provides a novel and non-obvious inventive solution to address these deficiencies.

It is to be understood that one feature of the software and system of the invention is the ability to provide rapid comparisons, value calculations, and/or visual representations of patients' brain MRI images, which could not be achievable manually and without the use of computers. Further, it is to be understood that, although in some embodiments, a display to show MRI image data comparison results is included in the system, this element is not required. In other words, the output by the software can be accomplished by any known means, including creation of a text file or similar output, textual representation on a computer screen or similar device, presentation of a graphical image of the subject's brain or portion thereof, highlighting determined abnormal region(s) or structure(s), a plot comparing numerical results to normal or a combination of these.

In another aspect, the invention provides a method of diagnosing a developmental disorder of a brain. In general, the method comprises using the software or the system of the invention to detect clinical differences in brain anatomy/morphology of a subject compared to a distribution of normal subjects. In exemplary embodiments, the method comprises comparing MRI image data of the brain of a first subject to MRI image data of a group of subjects, and determining if the brain of the first subject has an anatomical or morphological difference as compared to the group of subjects. In preferred embodiments, the comparison is made between MRI image data of a first subject and “normalized” MRI image data, which represents a “normal” brain for the developmental stage/age/gender of the first subject. If the comparison shows one or more abnormal anatomical features or morphologies, the subject is further investigated as potentially having a developmental disorder of the brain. The method of diagnosing has particular utility in assessing the brains of children, such as from newborns to children 18-22 years of age. In a further aspect, the invention provides a method of treating a subject having a developmental disorder of the brain. In general, the method comprises obtaining the results of a diagnostic method according to the invention, which indicates that a subject's brain may be undergoing, or has undergone, a developmental disorder, and treating the patient to reduce or eliminate the clinical and, preferably biochemical and/or physiological, effects of the disorder. As numerous developmental disorders are known in the art, as are methods to treat them, the skilled artisan in the medical field is able to practice this aspect of the invention without a detailed description of each disorder and the respective treatment regimens. The MRI analysis software may be used to compare MRI image data of the brain of a first subject to prior scans, and determine whether the brain is changing compared to prior scans to determine if a treatment is effective.

The MRI analysis software tool thus allows for accurate, precise, quantitative and automated pediatric brain MRI readings in the clinic. The MRI analysis software provides diagnostic information to help “fingerprint” neurological disorders at lower cost and higher reproducibility than visual inspection of the images. Quantitative evaluation by the software can help diagnose numerous diseases including for example neuroanatomical effects of prematurity, early hydrocephalus, ADHD and traumatic brain injury. In particular, early diagnosis of autism may be determined by the software, which will also reduce misdiagnosis rates.

As will be apparent to the skilled artisan, the invention encompasses a non-transitory computer-readable medium on which the software of the invention is stored or contained. The non-transitory medium can be any such medium known to the skilled artisan, including, but not limited to: an optical medium, such as a CD or DVD; a hard drive; a flash drive; a tape drive; or other reading and/or writing system that is coupled to a processor, may be used for the non-transitory medium. While the machine-readable medium is shown in an example to be a single medium, the term “machine-readable medium” should be taken to include a single medium or multiple media (e.g., a centralized or distributed database, and/or associated caches and servers) that store the one or more sets of instructions. The term “machine-readable medium” can also be taken to include any medium that is capable of storing, encoding, or carrying a set of instructions for execution by the machine and that cause the machine to perform any one or more of the methodologies of the various embodiments, or that is capable of storing, encoding, or carrying data structures utilized by or associated with such a set of instructions. The term “machine-readable medium” can accordingly be taken to include, but not be limited to, solid-state memories, optical media, and magnetic media.

In general, the hardware and software of the system 100 are designed with the foreknowledge of the hardware and software, such that the practitioner can easily implement suitable code for a given hardware platform. For example, where a state-of-the art, commercially available desktop computer is intended as the hardware, and an industry-standard operating system is run on the computer (e.g., Microsoft Windows, Linux, Mac OS), the software can be written in any appropriate language (e.g., Matlab, Python, C, C++) and compiled to run on the appropriate hardware/operating system.

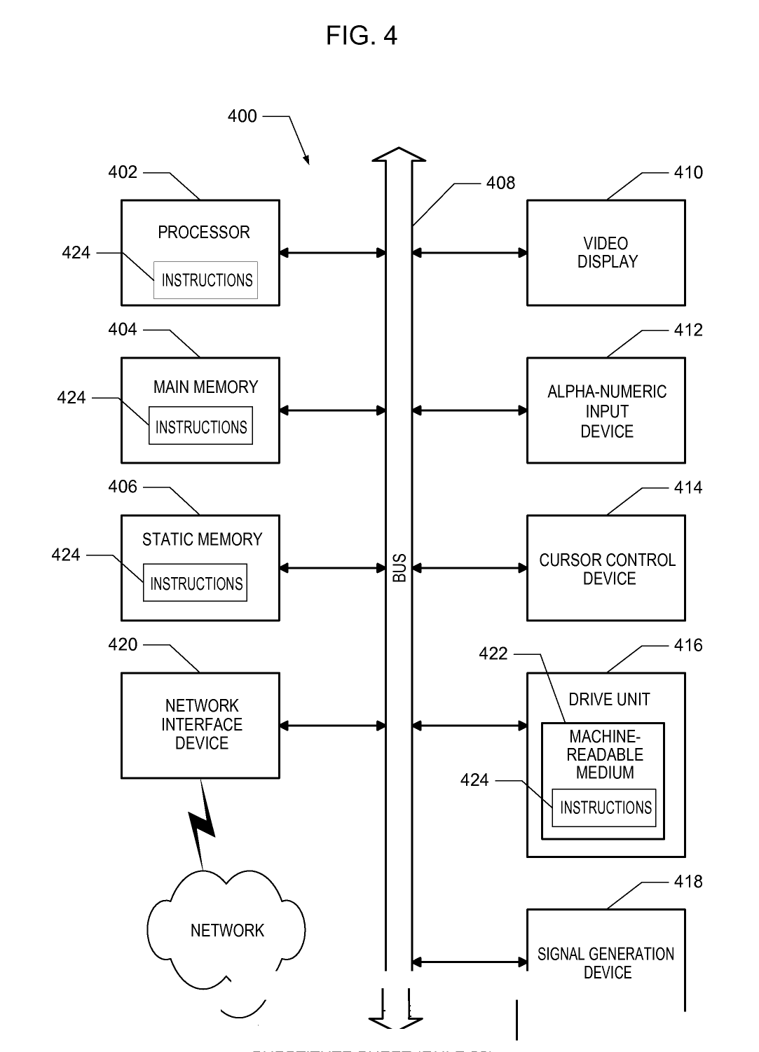

FIG. 4 shows an example computer system 400 may be used for the workstation 110 in FIG. 1 and includes a processor 402 (e.g., a central processing unit (CPU), a graphics processing unit (GPU), or both), a main memory 404, and a static memory 406, which communicate with each other via a bus 408. The computer system 400 may further include a video display unit 410 (e.g., a liquid crystal display (LCD) or a cathode ray tube (CRT)). The computer system 400 also includes an input device 412 (e.g., a keyboard), a cursor control device 414 (e.g., a mouse), a disk drive unit 416, a signal generation device 418 (e.g., a speaker), and a network interface device 420.

The disk drive unit 416 includes a machine-readable medium 422 on which is stored one or more sets of instructions (e.g., software 424) embodying any one or more of the methodologies or functions described herein. The instructions 424 may also reside, completely or at least partially, within the main memory 404, the static memory 406, and/or within the processor 402 during execution thereof by the computer system 400. The main memory 404 and the processor 402 also may constitute machine-readable media. The instructions 424 may further be transmitted or received over a network via the network interface device 420.

Furthermore, the system 100 may be conveniently implemented using one or more general purpose computer systems, microprocessors, digital signal processors, micro-controllers, application specific integrated circuits (ASIC), programmable logic devices (PLD), field programmable logic devices (FPLD), field programmable gate arrays (FPGA), and the like, programmed according to the teachings as described and illustrated herein, as will be appreciated by those skilled in the computer, software, and networking arts. The processor 402 may include a plurality of microprocessors including a master processor, a slave processor, and a secondary or parallel processor. The processor 402 comprises one or more controllers or processors and such one or more controllers or processors need not be disposed proximal to one another and may be located in different devices or in different locations.

In addition, two or more computing systems or devices may be substituted for any one of the computing systems in the system 100. Accordingly, principles and advantages of distributed processing, such as redundancy, replication, and the like, also can be implemented, as desired, to increase the robustness and performance of the devices and systems of the system 100. The system 100 may also be implemented on a computer system or systems that extend across any network environment using any suitable interface mechanisms and communications technologies including, for example telecommunications in any suitable form (e.g., voice, modem, and the like), Public Switched Telephone Network (PSTNs), Packet Data Networks (PDNs), the Internet, intranets, a combination thereof, and the like.

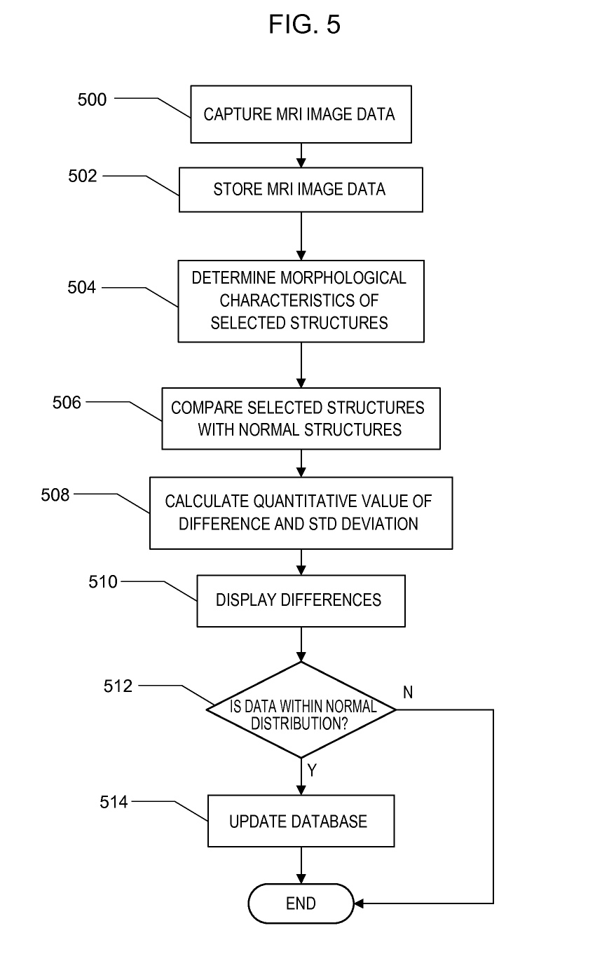

The process of the software on the example system 100 will now be described with reference to FIGS. 1-4 in conjunction with the flow diagram shown in FIG. 5. The flow diagram in FIG. 5 is representative of example machine readable instructions for analysis of MRI images. In this example, the machine readable instructions comprise an algorithm for execution by: (a) a processor, (b) a controller, and/or (c) one or more other suitable processing device(s) such as a CPU. The algorithm may be embodied in software stored on tangible media such as, for example, a flash memory, a CD-ROM, a floppy disk, a hard drive, a digital video (versatile) disk (DVD), or other memory devices, but persons of ordinary skill in the art will readily appreciate that the entire algorithm and/or parts thereof could alternatively be executed by a device other than a processor and/or embodied in firmware or dedicated hardware in a well-known manner (e.g., it may be implemented by an application specific integrated circuit (ASIC), a programmable logic device (PLD), a field programmable logic device (FPLD), a field programmable gate array (FPGA), discrete logic, etc.). For example, any or all of the components of the interfaces could be implemented by software, hardware, and/or firmware. Also, some or all of the machine readable instructions represented by the flowchart of FIG. 5 may be implemented manually. Further, although the example algorithm is described with reference to the flowcharts illustrated in FIG. 5, persons of ordinary skill in the art will readily appreciate that many other methods of implementing the example machine readable instructions may alternatively be used. For example, the order of execution of the blocks may be changed, and/or some of the blocks described may be changed, eliminated, or combined.

In FIG. 5, the system 100 first captures MRI image data through the MRI device 102 (500). The MRI image data is cataloged along with relevant patient data and stored in a storage device such as the hospital PAC 106 (502). The stored images are then analyzed to determine morphological characteristics of the whole brain or selected structures of the brain (504). The selected structures are compared with normal structure determined from the database 112 (506). The quantitative value of the differences and the standard deviation between the selected structures and the normal structures is computed using comparison and statistical routines (508). The differences are displayed in the form of an interface such as the interface 200 in FIG. 2 (510). The system 100 determines whether the collected data is within the normal (512). If the stored data is normal, the relevant data from the subject is then stored in the database 112 (514). If the stored data is outside the normal, e.g., outside of one standard deviation, this may indicate an abnormal region which requires further investigation by the radiologist/neurologist, and the display will show such a potential abnormality on an interface such as the interface 200 in FIG. 2.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only.

What is claimed is:

1. A method for analyzing a brain of a subject, said method comprising:
acquiring MRI image data of the brain or portion of the brain of the subject;
determining one or more morphological characteristics of one or more structures of the brain;
comparing the one or more morphological characteristics to the same one or more morphological characteristics of the structure of a normal brain;
calculating a quantitative value of the difference between the determined one or more morphological characteristics and the same one or more morphological characteristics of the normal brain; and
outputting the quantitative value in a format for a health care provider.

2. The method of claim 1, wherein the quantitative value is a statistical measurement value.

3. The method of claim 2, wherein the statistical measurement value is a number of standard deviations between the quantitative data and the normal brain.

4. The method of claim 3, wherein the outputting includes displaying an image of the brain of the subject in which one or more abnormal areas determined by the value of the standard deviation is highlighted.

5. The method of claim 3, wherein the outputting includes displaying an image of a structure of the brain including an area of abnormal growth or development determined by the value of the standard deviation being highlighted.

6. The method of claim 1, wherein the outputting includes providing a textual report including data associated with the quantitative value.

7. The method of claim 6, wherein the data includes at least one of the group of head circumference, brain circumference, whole brain volume, grey matter volume, white matter volume, and ventricular volume.

8. The method of claim 1, wherein the one or more morphological characteristics of the structure of a normal brain is accessed from a database, the method further comprising updating the database with data from the subject.

9. The method of claim 1, further comprising detecting one or more clinical differences between the one or more morphological characteristics to the one or more morphological characteristics from previous image data from the brain of the subject.

10. The method of claim 1, further comprising:
obtaining age and metric data of the subject; and
determining a developmental disorder via analysis of the quantitative value of the difference between the determined one or more morphological characteristics and the same one or more morphological characteristics, the age data and the metric data of the subject.

11. A system for analyzing a brain of a subject, said system comprising:
a storage device including MRI image data of the brain or portion of the brain of the subject;
a database including data from a normal brain; and
a controller coupled to the storage device and the database, the controller:
determining one or more morphological characteristics of one or more structures of the brain;
comparing the one or more morphological characteristics to the same one or more morphological characteristics of the structure of a normal brain from the data from the normal brain; and
calculating a quantitative value of the difference between the determined one or more morphological characteristics and the same one or more morphological characteristics of the normal brain.

12. The system of claim 11, further comprising a display coupled to the controller, the display displaying the quantitative value.

13. The system of claim 11, wherein the controller determines a value of the standard deviation based on the quantitative value and an area of abnormal growth or development is determined based on the standard deviation.

14. The system of claim 13, wherein the display provides a two-dimensional or three-dimensional image of the subject's brain from the MRI image data, the image including the area of abnormal growth or development being highlighted.

15. The system of claim 12, wherein the display provides quantitative comparison data between the subject's brain and a normal brain.

16. The system of claim 12, wherein the display includes an image of a structure of the brain including the area of abnormal growth or development being highlighted.

17. The system of claim 12, wherein the quantitative comparison includes at least one of the group of head circumference, brain circumference, whole brain volume, grey matter volume, white matter volume, and ventricular volume.

18. The system of claim 11, further comprising an MRI scanner coupled to the storage device.

19. A method of treating a subject having a developmental disorder of the brain, the method comprising:
acquiring MRI image data of the brain or portion of the brain of the subject;
determining one or more morphological characteristics of one or more structures of the brain;
comparing the one or more morphological characteristics to one or more of the same morphological characteristic of the structure of a normal brain;
calculating a quantitative value of the difference between the determined one or more morphological characteristics and the one or more same morphological characteristics of the normal brain;
determining the subject's brain is undergoing, or has undergone, a developmental disorder based on the quantitative value; and
treating the patient to reduce or eliminate the clinical effects of the disorder.

20. The method of claim 21, wherein the treating further includes treating the biochemical and/or physiological, effects of the disorder.

21. A non-transitory, machine readable medium having stored thereon instructions for analyzing a brain of a subject, the stored instructions comprising machine executable code, which when executed by at least one machine processor, causes the machine to:
acquire MRI image data of the brain or portion of the brain of the subject;
determine one or more morphological characteristics of one or more structures of the brain;
compare the one or more morphological characteristics to the same one or more morphological characteristics of the structure of a normal brain;
calculate a quantitative value of the difference between the determined one or more morphological characteristics and the one or more morphological characteristics of the normal brain; and
output the quantitative value in a format for a health care provider.
